(19) United States
(12) Patent Application Publication
(10) Pub. No.: US 2025/0208098 A1
(43) Pub. Date: Jun. 26, 2025

(54) METHOD OF CHARACTERIZING A TARGET OBJECT IN A MEDIUM

(71) Applicants: SUPERSONIC IMAGINE, Aix-en-Provence (FR); CENTRE NATIONAL DE LA RECHERCHE SCIENTIFIQUE (CNRS), Paris (FR); Ecole Supérieure de Physique et de Chimie Industrielles de la Ville de Paris, Paris (FR); UNIVERSITÉ GUSTAVE EIFFEL, Marne la Vallée CEDEX 2 (FR)

(72) Inventors: Arthur LE BER, Paris (FR); Antton GOICOECHEA, Paris (FR); William LAMBERT, Aix-en-Provence (FR); Alexandre AUBRY, Paris (FR); Xiaoping JIA, Paris (FR); Arnaud TOURIN, Paris (FR); Mathias FINK, Paris (FR); Nicolas ETAIX, Aix-en-Provence (FR); Stefan ROTTER, Vienna (AT); Lukas RACHBAUER, Vienna (AT)

(73) Assignees: SUPERSONIC IMAGINE, Aix-en-Provence (FR); CENTRE NATIONAL DE LA RECHERCHE SCIENTIFIQUE (CNRS), Paris (FR); Ecole Supérieure de Physique et de Chimie Industrielles de la Ville de Paris, Paris (FR); UNIVERSITÉ GUSTAVE EIFFEL, Marne la Vallée CEDEX 2 (FR)

(21) Appl. No.: 18/987,016

(22) Filed: Dec. 19, 2024

(30) Foreign Application Priority Data
Dec. 21, 2023 (FR) FR2314789

(51) Int. Cl. G01N 29/22; G01N 29/06; G01N 29/44

(52) U.S. Cl. CPC G01N 29/221; G01N 29/069; G01N 29/4472; G01N 29/4481

(57) ABSTRACT

This disclosure relates to a method for characterising a target object in a medium. The method comprises: obtaining a reference matrix F(q) associated with the target object, measuring a reflection matrix R associated with the probed medium, and projecting the reflection matrix R onto the reference matrix F(q) to estimate a probability P(q) that the object is in a state q.

### PRIOR ART

Ultrasound imaging provides a non-invasive method of viewing the internal structure of a material, animal or human body or parts thereof (organs, for example).

Examples of different ultrasound imaging modes may include a B-mode (i.e. brightness mode), a Doppler mode or a ShearWave® Elastography mode (i.e. shear wave elastography mode).

In ultrasound imaging, an array of piezoelectric transducers is typically placed opposite the medium to be imaged. Those elements can emit/receive ultrasound waves independently over a wide spectral band. From a series of insonifications of the medium by the array, the echoes backscattered by the heterogeneities of the medium are recorded by those same transducers or by others. The signals thus recorded are then stored in a response matrix. The matrix can be acquired for different types of incident waves.

A basic acquisition sequence consists of emitting each incident wave with one element at a time and, for each emission, recording the field reflected by the medium on all the elements in the array. This canonical base, or transducer base, was first used to describe the principle of iterative time reversal, see the “DORT” method described by: Claire Prada, Mathias Fink, “Eigenmodes of the time reversal operator: A solution to selective focusing in multiple-target media,” Wave Motion, Volume 20, Issue 2, 1994, Pages 151-163, ISSN 0165-2125. This canonical base can be used in the field of non-destructive testing, where it is known as “full matrix capture”.

In addition, there are ultrasound imaging technologies that use coherent plane-wave compounding for very high frame rate ultrasonography and transient elastography, see Montaldo G, Tanter M, Bercoff J, Benech N, Fink M. Coherent plane-wave compounding for very high frame rate ultrasonography and transient elastography. IEEE Trans Ultrason Ferroelectr Freq Control. 2009 Mar;56(3):489-506. doi: 10.1109/TUFFC.2009.1067. PMID: 19411209.

In this context, FR3114159 describes a method of ultrasound characterisation of a medium comprising a step of generating a series of incident ultrasound waves, a step of generating an experimental reflection matrix Rᵤᵢ(t) defined between the input emission base (i) and an output reception base (u), a step of determining a focused reflection matrix Rᵣᵣ=[R(rᵢₙ, rₒᵤₜ, δt)] of the medium between an input virtual transducer (TVᵢₙ) calculated from an input focus of the experimental reflection matrix and an output virtual transducer (TVₒᵤₜ) calculated from an output focus of the experimental reflection matrix, the responses of the output virtual transducer (TVₒᵤₜ) being taken at a time instant offset by an additional time delay δt relative to a time instant of the responses of the input virtual transducer (TVᵢₙ). This focused reflection matrix plays a pivotal role in the development of matrix ultrasound imaging to quantify and correct aberration and multiple scattering issues in ultrasound imaging.

### Disclosure Statement

The method and system described herein relate to technologies used to detect, locate and characterise a target object, particularly objects that are difficult to image, for example objects in a scattering medium. This description applies in particular, but not exclusively, to medical or veterinary imaging for the location and characterisation of targets in biological media (e.g. micro-calcifications, bubbles, needles or lesion markers in ultrasound imaging). It can also be applied to the detection of defects useful for non-destructive testing in industry. Although the experimental demonstrations presented in this document are carried out in reflection in the field of ultrasound imaging, this disclosure can also be applied in emission to all fields of wave physics (e.g. radar technologies, microscopy, seismology) for which one or more multi-element arrays make it possible to measure the response matrix of a medium in reflection and/or emission of the latter.

In particular, it may be desirable to more reliably detect the position and/or orientation of a target object in a medium of interest. For example, if the target object is a medical device (e.g. a marker) implanted in a human body, it is desirable to accurately locate its position in the human tissue, for example as part of a surgical procedure such as the removal of a tumour or suspicious mass.

According to a more general example, it may be possible to characterise a target object in a medium, for example, not only to locate its position, but also to obtain other information about the object or its environment (temperature, pressure, etc.), for example its shape and/or the structure of the object, the size of the object, the composition of the object, and/or to monitor changes in those parameters (nature, structure, composition) over time.

In another embodiment, the state of the object can be used to characterise the surrounding medium (pressure, temperature, mechanical properties, etc.), the response of the object also depending on the medium surrounding the region of interest.

As explained in more detail below, conventional techniques (e.g. mode B ultrasound imaging) often do not allow accurate detection or characterisation of a target object, for example due to the presence of a scattering medium generating ultrasound ‘speckle’. “Speckle” can be seen as a granular profile which can vary from one part of the medium to another. “Speckle” can be explained by the coherent formation of the echo from numerous small scatterers in the medium. Some objects are also difficult to image or are even undetectable using conventional imaging techniques due to their geometry or their acoustic or mechanical properties.

Thus, a method for characterising a target object in a medium is provided according to a particular embodiment of the disclosure. The method comprises: obtaining a reference matrix F(q) associated with the target object (for example associated with a state q of the target object), obtaining a reflection matrix R of the medium, and projecting the reflection matrix R onto the reference matrix F(q) to estimate a probability P(q) that the object is in a state q.

Therefore, as disclosed in this application, considering that a reflection matrix of the medium (and hence of the target object contained therein) is obtained, and that the reflection matrix can be projected onto a reference matrix of the target object (i.e. which “describes” the target object), the target object can be reliably and accurately characterised in the support, irrespective of any disturbance factors of the medium.

In other words, the reference matrix can be designed, predetermined and used to search a medium of interest and/or detect the target object more reliably and accurately.

The reference matrix F(q) can therefore be used to characterise the target object.

For example, the medium may be breast tissue containing a marker. Breast tissue typically causes ultrasound speckle. The presence of a target object (a marker, for example) is difficult to detect on a conventional B-mode ultrasound image. However, the use of a reference matrix F(q) associated with the target object allows detection with excellent contrast and location with an accuracy well below the diffraction limit of the medium.

According to a particular embodiment, the target object comprises at least one of: a predefined element of the medium, such as a tissue of the medium, for example a muscle fibre, and an external object inserted into the medium, such as a medical device, for example a biomarker, all or part of a biopsy device, a needle or a probe.

According to a particular embodiment, the state q of the object comprises at least one parameter from among: the relative position and/or relative orientation of the object in the medium, the orientation of the object in the medium, the shape and/or structure (initial and/or current) of the object, the size of the object, the composition of the object, local intensive parameters of the surrounding medium, such as temperature, pressure, composition, mechanical properties and/or the concentration of a compound of the surrounding medium.

In a particular embodiment, the state q of the object can be measured at different times and can therefore be characterised dynamically.

For example, obtaining the reflection matrix may involve obtaining reflection matrices measured at different times t. Each of the reflection matrices can be projected onto the reference matrix to measure the dynamics of the state of the object.

Consequently, the estimated probability that the target object is in a particular state can provide information about the characteristics of the object or its medium. For example, if we estimate with a high probability that the object has a specific size and is in a specific position, that size and position can characterise the object in part or in full.

The state of the target can also be expressed as a matrix Q: Each column qᵢ(in vector form) of the Q matrix can correspond to a state parameter of the target object, for example q₁: Position, q₂: Orientation, etc.

According to a particular embodiment, obtaining a reference matrix F(q) comprises obtaining a dictionary of reference matrices F(q) associated with the target object.

The matrix dictionary can be used, for example, to exploit the matrix signature of a target object to characterise it precisely, for example to detect it with excellent contrast in a medium that generates speckle when it is insonified, for example by ultrasound waves.

According to a particular embodiment, projecting the reflection matrix R comprises: projecting the reflection matrix R onto the set of reference matrices F(q) to estimate a probability P(q) that the object is in a state q.

According to a particular embodiment, projecting the reflection matrix R comprises applying a matrix filter to the reflection matrix R.

According to a particular embodiment, the matrix filter comprises one or more standard dot products between the reflection matrix R and the set of reference matrices F(q), or the matrix filter comprises an artificial intelligence model.

The standard dot product can also be a correlator and/or a mathematical function. The result of this mathematical function can be the estimated probability.

The artificial intelligence model is a neural network, for example.

According to a particular embodiment, the reflection matrix R is a reflection matrix expressed in the focused base or in another base, such as plane wave bases, transducer bases, etc. The input data for the artificial intelligence model may comprise the reflection matrix R and the reference matrix F(q). The result of the artificial intelligence model can comprise the estimated probability. The model can be pre-trained in the task of estimating the probability.

According to a particular embodiment, the reference matrices F(q) in the dictionary depend on a reference state of the target object or its medium, the state optionally of at least one of: position, orientation, shape, structure, size, composition and/or local intensive parameters of the surrounding medium such as temperature, pressure or the concentration of a compound.

According to a particular embodiment, obtaining the dictionary of reference matrices F(q) comprises: a measurement (for example of an experimental type) of a first reference matrix corresponding to a reflection matrix R₀(q₀) for a first state q₀ of the target object, and/or a measurement (for example of an experimental type) of a first set of reference matrices for a first set of states q of the target object and/or associated with several states q of the target object.

Principally, a reference matrix may correspond to or may be a reflection matrix according to this disclosure. However, the reference matrix has a specific function, namely to serve as a reference (for example in a dictionary). However, the manner in which it is obtained may be the same, for example as part of a measurement.

The difference between the reference matrix and the reflection matrix may therefore lie in the context in which it is measured. While a reference matrix according to this disclosure may be measured during an experiment in order to obtain a reference of a target object, the reflection matrix according to this disclosure may be measured on a particular support (e.g. a patient) during deployment.

Therefore, the reference matrix or matrices can be obtained during the preparation of the method or system and stored in advance, the reflection matrix can be obtained “on the fly” (e.g. in real or near-real time) during the deployment of the method or system. The reflection matrix can also be projected onto the reference matrix “on the fly” (in real or near-real-time, for example).

According to a particular embodiment, obtaining the reference matrix dictionary F(q) comprises: numerically calculating a second reference matrix for a second state q of the target object from the first reference matrix, for example measured for a state q₀ of the target object.

According to a particular embodiment for which the state q is the position r of the target object, the second reference matrix is simulated numerically from the first reference matrix measured for a first position r₀ of the object by a virtual translation of the target object into a new position r, this operation optionally being carried out in a focused base, the transducer base, or in the plane wave base.

The second reference matrix can be simulated numerically, for example using a model of wave propagation from a transducer array to the target object and a wave scattering model by the target object.

The choice of the base in which the virtual translation should be carried out may be dictated by the need to minimise the resulting computational cost.

According to a particular embodiment, the reference matrix dictionary comprises the first and second reference matrices.

According to a particular embodiment, the reference matrix dictionary is adapted/adjusted/optimised using at least one of the following methods:
adaptation of the matrix filter depending on the first reference matrix in order to make the matrix filter more specific to the sought target object relative to the surrounding medium;
optimisation of the dictionary's frequency spectrum to improve the contrast between the target object and a noise associated with the medium;
and construction of the dictionary from an estimator R₀⁻¹(q₀) of the inverse matrix of the first reference matrix R₀(q₀).

According to a particular embodiment, the first measured reference matrix R₀(q₀) and/or the reflection matrix R is obtained by an ultrasound imaging and/or echography method.

According to a particular embodiment, obtaining the first measured reference matrix and/or the reflection matrix comprises:—a step of generating a series of incident waves (USin) in a medium, by means of an array (10) of transducers (11), said series of incident waves being an emission base (i); and—a step of generating an experimental reflection matrix Rᵤᵢ(t) defined between the input emission base (i) and an output receiver base (u) (i.e. a transducer base) for an echo time t;—a step of determining a focused reflection matrix Rᵣᵣ=[R(rᵢₙ, rₒᵤₜ, δt)] being a focused base, the focused reflection matrix Rᵣᵣ comprising responses of the medium R(rᵢₙ, rₒᵤₜ, St) between a virtual input transducer (TVᵢₙ) of spatial position rᵢₙ and a virtual output transducer (TVₒᵤₜ) of spatial position rₒᵤₜ, the responses of the virtual output transducer (TVₒᵤₜ) being taken at a time instant offset by an additional time delay St relative to a time instant of the responses of the input virtual transducer (TVᵢₙ).

The waves may include ultrasound waves.

The focused reflection matrix can be obtained numerically using a beamforming process.

According to a particular embodiment, obtaining a reference matrix F(q) comprises: creating a reference matrix dictionary in a fully synthetic manner by numerically simulating a first reference matrix of a virtual reflector (e.g. a reflectivity mirror of orientation α and size l_{c}.) for a first state q₀, and numerically calculating a second reference matrix for a new state q of the target object from the first reflection matrix R₀(q₀).

The reference matrix dictionary can therefore also be built entirely synthetically, i.e. without experimental measurements.

This disclosure also relates to a method of recognising a target object in a medium, the method comprising a method according to any of the preceding aspects.

This disclosure also relates to a method for generating a map of a medium comprising a target object, comprising a method according to any of the preceding aspects, and/or generating the map, in particular depending on the estimated state of the target object.

According to a particular embodiment, the map of the medium comprises an image of the medium (for example obtained by a predefined method) and an identification of the target object superimposed thereon, wherein the identification of the target object is determined by a method according to one of the preceding aspects.

For example, a point at the location of the target object or an image (which may be a schematic image or a photograph) of the target object is superimposed on the image of the medium at the corresponding location.

This disclosure also relates to a computer program containing instructions which, when the program is run by a computer, cause the computer to implement the method according to any of the preceding aspects.

This disclosure, according to a particular embodiment, also relates to a system for characterising a target object in a medium. The system comprises a processor configured to:
obtain a reference matrix F(q) associated with the target object,
obtain a reflection matrix R of the medium,
project the reflection matrix R onto the reference matrix F(q) to estimate a probability P(q) that the object is in a state q.

The system may have functions that correspond to the operations of the method according to the present disclosure.

For example, the system may be a computer which may be associated with an ultrasound imaging system. The system may also be an ultrasound imaging system.

The characteristics and advantages of the disclosure will become apparent from the following description, which is given solely by way of non-limiting example and with reference to the attached figures. In particular, the examples illustrated in the figures can be combined unless there is a clear inconsistency.

### BRIEF DESCRIPTION OF THE FIGURES

Other characteristics and advantages of this disclosure will be apparent from the description of the particular and non-limiting embodiments of this disclosure below, with reference to appended FIGS. 1 to 12, in which:

FIG. 1 schematically shows a method for characterising a target object in a medium according to examples in this disclosure.

FIG. 2 shows a schematic drawing of an ultrasound imaging system according to examples in this disclosure.

FIG. 3 schematically shows the principle of a method for decomposing the time reversal operator according to an example.

FIG. 4 schematically shows a diagram summarising the various stages of the matrix projection method based on a calibration measurement of the target object's reflection matrix according to examples in this disclosure.

FIG. 5 schematically shows a proof of concept according to a first experiment comprising matrix imaging of steel spheres in highly scattering granular media according to examples in this disclosure.

FIG. 6 schematically shows a device and a method for obtaining a reference matrix R₀(q₀) measured experimentally in the context of the first experiment according to examples in this disclosure. It also describes the information contained in this matrix in terms of its spatial and temporal components.

FIG. 7 schematically shows a method for detecting and locating a lesion marker in the ultrasound speckle according to a second experiment, according to examples in this disclosure.

FIG. 8 schematically shows an example of matrix mapping of calf muscle fibres, in which the reference matrix dictionary is constituted entirely synthetically in accordance with this disclosure

FIG. 9 schematically shows the virtual translation of the target object position according to examples in this disclosure.

FIG. 10 schematically shows the emission and reception of a plane wave associated with an angle of incidence from which the data must be filtered in matrix R(q=q₀) according to examples in this disclosure.

FIG. 11 schematically shows position likelihood maps for each target resulting from matrix projection according to examples in this disclosure.

FIG. 12 schematically shows a spectrum of singular values σᵢ of matrices R(qᵢ=q₀) associated with the spheres according to examples in this disclosure.

### DESCRIPTION OF THE EMBODIMENTS

In the various figures, which are provided by way of illustration, the same numerical references designate identical or similar elements, unless there is an obvious inconsistency.

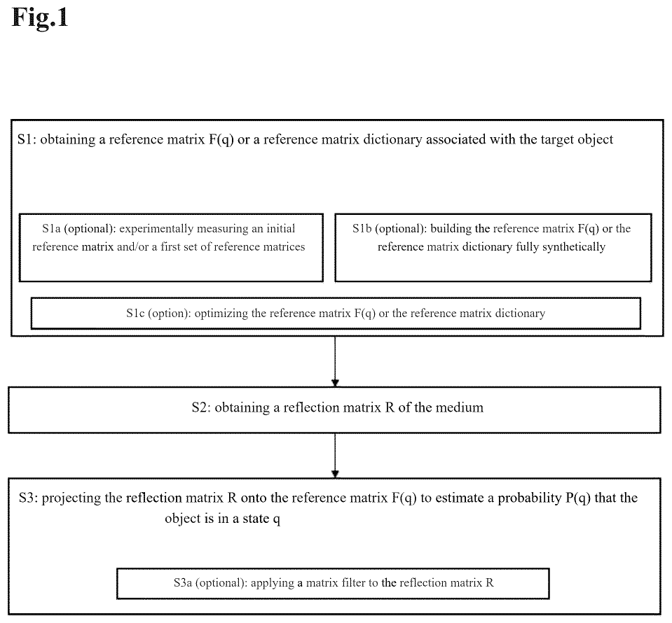

FIG. 1 schematically shows a method for characterising a target object in a medium according to examples in this disclosure.

The target object may comprise, for example, a predefined element of the medium or region of interest, such as a tissue of the medium and/or a biocompatible or non-biocompatible object inserted into the medium, such as a medical device, for example a marker, such as a marker used in surgery, a biopsy device, a needle or a probe.

The state q of the target object may include, for example, the position of the object in the medium, the orientation of the object in the medium, the shape and/or (current) structure of the object, the size of the object, the composition of the object, or local intensive parameters of the medium such as temperature, pressure or the concentration of a compound of the medium surrounding the region of interest and/or a variation in the state of the object.

In a particular embodiment, the state q of the object can be measured at different times and can therefore be characterised dynamically.

The method according to examples in this disclosure may be an ultrasound imaging method. However, other types of imaging or scanning methods are possible, as is described below.

The method can be used, for example, to recognise a target object in a medium.

The method can also be used to generate a map of a medium containing a target object or likely to contain a target object. According to a particular embodiment, the mapping of the medium consists of an image of the medium (obtained by a predefined method, for example) and an identification of the superimposed target object (a probability map of the presence of the target object, for example), wherein the identification of the target object is determined by a method as described below. For example, a point at the location of the target object or an image (which may be a schematic image) of the target object superimposed on the image of the medium.

The method comprises an operation S1 to obtain a reference matrix F or a reference matrix dictionary F(q) associated with a state q of the target object.

The reference matrices F of the dictionary can depend on a reference state of the target object, the state optionally from at least one parameter from among: the position, orientation, shape and/or structure, size, composition of the object or local intensive parameters of the medium such as temperature, pressure or the concentration of a compound of the surrounding medium.

According to an optional operation S1a, obtaining the reference matrix F(q) may comprise experimentally measuring a first reference matrix known as the reflection matrix R₀(q₀) for a state q₀ of the target object, and/or experimentally measuring a first set of reference matrices for a first set of states q.

In addition, operation S1a may comprise obtaining a second reference matrix for further values of the state q of the target object depending on the first reference matrix. For example, if state q is position r of the target object, the second reference matrix can be simulated numerically from the first reflection matrix measured for a position r₀ by translating the target object to a new position r.

This virtual translation of the object can be carried out in a new qstate, optionally in a focused base, the transducer base or the plane wave base.

The reference reflection matrix R₀(q) can also be simulated numerically, for example using a wave propagation model from a transducer array to the target object and a wave scattering model by the target object.

According to an optional operation S1 b (taken as an alternative to operation S1a or combined with it), obtaining a reference matrix F(q) may comprise synthetically building a reference matrix dictionary (i.e. obtained using simulations and/or calculations). For example, a first reflection matrix of a virtual reflector in a predefined state (a scattering mirror the states of which are position r₀, orientation α and size l_{c}.) can be numerically simulated. A second reference matrix R₀(q) for a second state q of the target object (e.g. at another position r of the mirror) can be numerically simulated depending on the first reference matrix.

The reference matrix dictionary can also be built entirely synthetically, i.e. without any experimental measurements.

According to an optional operation S1 c, the reference matrix F(q) or the reference matrix dictionary can be optimised using at least one of the following methods:
* adapting the matrix filter depending on the first reference matrix in order to make the matrix filter more specific to the sought target object relative to the surrounding medium;
* optimising the dictionary's frequency spectrum to improve the contrast between the target object and a noise associated with the medium;
* building the dictionary from an estimator R₀⁻¹(q) of the inverse matrix of the first reference matrix R₀(q).

In an operation S2, reflection matrix R of the medium can be obtained using an ultrasound echography method, for example as described in file FR3114159.

According to a particular embodiment, obtaining the reference matrix (i.e. experimentally measuring a first reference matrix and/or a first set of reference matrices) and/or obtaining the reflection matrix comprises:
a step of generating a series of incident waves (USin) in a medium by means of an array (10) of transducers (11), said series of incident waves being an emission base (i); and
a step of generating an experimental reflection matrix Rᵤᵢ(t) defined between the input emission base i and an output reception base (u) (i.e. a transducer base);
a step of determining a focused reflection matrix Rᵣᵣ=[R(rᵢₙ, rₒᵤₜ, δt)] in a focused base, the focused reflection matrix Rᵣᵣ comprising responses R(rᵢₙ, rₒᵤₜ, St) of the medium between an input virtual transducer (TVᵢₙ) of spatial position rᵢₙ(rᵢₙ) and an output virtual transducer (TVₒᵤₜ) of spatial position rₒᵤₜ (rₒᵤₜ), the responses of the output virtual transducer (TVₒᵤₜ) being taken at a time instant offset by an additional time delay St relative to a time instant of the responses of the input virtual transducer (TVᵢₙ).

In an operation S3, reflection matrix R is projected onto reference matrix F(q) to estimate a probability P(q) that the object is in state q

In particular, projecting reflection matrix R can comprise projecting reflection matrix R onto the set of reference matrices F(q) in the dictionary to estimate a probability P(q) that the object is in state q.

According to an optional operation S3a, projecting reflection matrix R may comprise applying a matrix filter to reflection matrix R. This matrix filter comprises a standard dot product (or normalized scalar product) between reflection matrix R and the set of reference matrices F(q), or the matrix filter comprises an artificial intelligence model built using the reference matrices. In other words, the matrix filter can be a correlator (standard dot product) and/or a mathematical function.

The result of this correlator or mathematical function can be the estimated probability. The artificial intelligence model is a neural network, for example.

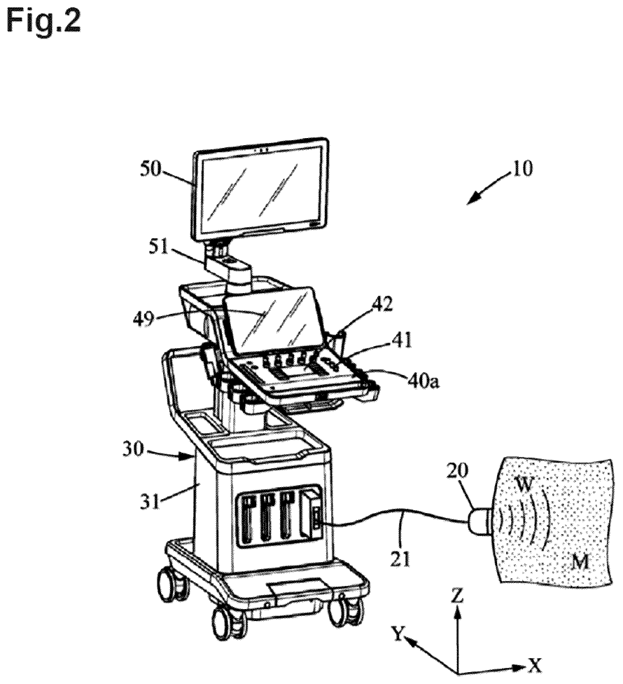

FIG. 2 shows a schematic drawing of an ultrasound imaging system 10 according to examples of the present disclosure. It should be noted that the system 10 according to this disclosure may also be a type of system other than an ultrasound imaging system, as illustrated below.

The system 10 may have functions that correspond to the operations of the method according to this disclosure.

The system can be configured to characterise a target object in a medium. The system comprises a processor configured to:
obtain a reference matrix F(q) associated with the target object,
obtain a reflection matrix R of the medium, and
project the reflection matrix R onto reference matrix F(q) to estimate a probability P((q) that the object is in state q

The ultrasound imaging system 10 may comprise:
a probe 20,
a processing unit 30 to process an image based on the signals received by the probe (corresponding, for example, to processing unit 11 in FIG. 1),
a control panel 40 connected to processing unit 30, said control panel comprising, for example, buttons 41 and a touch pad 42,
a screen 50 to view images.

Probe 20 can be connected to processing unit 30 using a cable 21 or using a wireless connection, and is capable of emitting ultrasound waves W into medium M and of receiving ultrasound waves W from medium M, said ultrasound waves resulting from reflections of said emitted ultrasound waves on scattering particles or scatterers inside said medium. Probe 20 may be formed from or contain a transducer array comprising a plurality of transducers, each of which converts an electric signal into a vibration and vice versa. A transducer is, for example, a piezoelectric element. The transducer array may have a hundred or more transducers. The transducer array is linear or curved and is often arranged through a lens on an external surface of medium M so as to be coupled to the medium and to vibrate and emit or receive ultrasound waves W.

Processing unit 30 may have receiving devices to amplify and/or filter the signals received from probe 20, and converters (analogue to digital converters and digital to analogue converters) to convert the signals into data representative of the signal. The data can be stored in a memory in the processing unit and/or directly processed to calculate intermediate data (beamforming data). Processing unit 30 can use any known processing method to process the image based on the signals received from the probe, such as beamforming. The processed ultrasound image data can be:
a simple image of the medium (reflectivity image of the medium obtained in B mode) generally in greyscale to view the organs inside the medium, or
an image showing a speed or flow in the medium (Doppler image), for example useful for viewing blood vessels in the medium, and/or
an image showing a mechanical characteristic of the medium (elasticity), for example useful for identifying tumours within the medium (for example shear wave elastography image data (ShearWave™ Elastography or SWE) as described below).

Display screen 50 can be a screen to view the image processed by processing unit 30.

The display screen can be articulated on a support arm 51 to improve positioning for the user.

Control panel 40a is, for example, a part of system housing 31, said part comprising a panel housing having a substantially flat surface tilted towards the user for one-handed operation.

Ultrasound probe 20 may comprise, for example, one or more transducer elements (not shown in FIG. 1), each configured to convert an electric signal received from system 10 into ultrasound waves. The transducer elements can be configured to emit (a) waves into the medium and/or to receive (b) a plurality of ultrasound signals from the medium, possibly in response to the emission (a).

System 10 may be a medical system, for example an ultrasound system. For example, the system can be associated with an ultrasound probe 20, in order to collect ultrasound data from a medium, for example composed of living tissue and/or in particular human or animal tissue.

However, system 10 can be any type of electronic system. For example, the system may also be a type of medical system other than an ultrasound imaging system. Consequently, probe 20 can be any type of imaging device or sensor using waves other than ultrasound waves (for example, waves with a wavelength different from the ultrasound wavelength and/or waves that are not sound waves).

Examples of medical imaging systems include an ultrasound imaging system, an X-ray imaging system (particularly for mammography) and an MRI (Magnetic Resonance Imaging) system.

In other examples, system 10 may comprise at least one processing unit (or processor) and in addition a memory (not shown). In examples, the processing unit and memory may be incorporated into the system or may be a computer or computing device communicatively linked thereto. Depending on the exact configuration and type of computing device, the memory (which stores the instructions used to assess the ultrasound data or run the processes described herein) may be volatile (such as RAM), non-volatile (such as RAM, flash memory, etc.) or a combination of both. In addition, system 10 may also include storage devices (removable and/or non-removable) including, but not limited to, magnetic or optical disks or tapes.

System 10 can be a single computer operating in a networked environment using logical connections with one or more remote computers. The remote computer may be a personal computer, a server, a router, a network PC, a peer device or other common network node, and typically includes many or all of the elements described above as well as others not mentioned. Logical connections may include any method supported by the available communication media. Such networking environments are common in offices, corporate computer networks, intranets and the Internet.

In addition, the processing unit can be configured to process data and/or send data to an external device, such as a display device, a server, a computer running an artificial intelligence (AI) algorithm, a dedicated workstation, or any other external device.

The following description summarises the technical and scientific background of this disclosure and illustrates the method of this disclosure with several concrete examples and experiments. Those example embodiments do not limit this disclosure, but are primarily to provide a better understanding, including of the advantages of this disclosure.

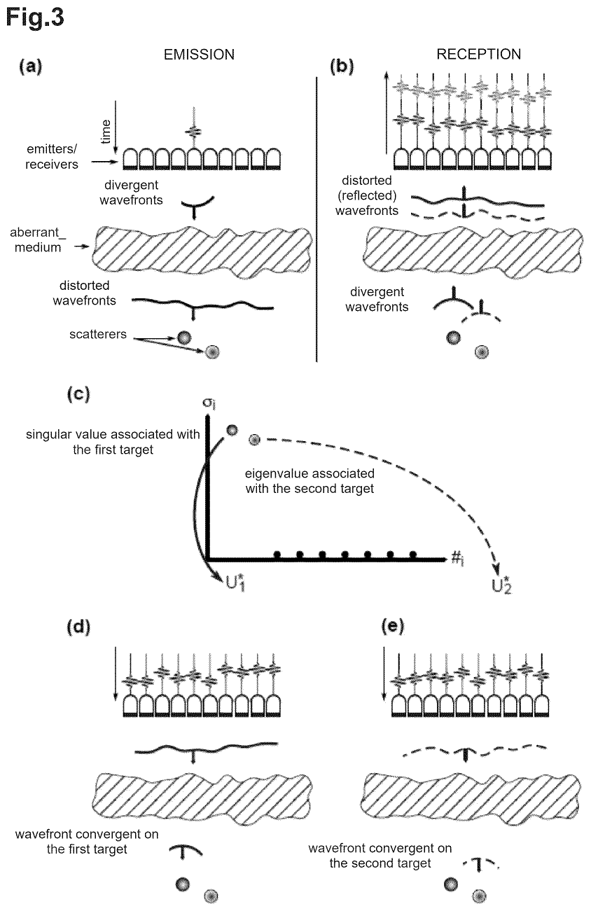

FIG. 3 schematically shows the principle of a method for decomposing the time reversal operator according to an example.

Part (a) of FIG. 3 schematically shows an incident wave which is emitted by one of the transducers and propagates through the medium to the target (left-hand diagram). Part (b) of FIG. 3 schematically shows that this wave is then reflected by the scatterers present in the medium under study and propagates again through the medium. The backscattered echoes are then recorded by the array of transducers. This operation is repeated for each array element acting as a source of wave emission. All the impulse responses between the transducers in the array are stored in reflection matrix R thus acquired in the canonical base.

Part (c) of FIG. 3 schematically shows the spectrum of singular values of matrix R. Parts (d) and (e) of FIG. 3 schematically show that each eigenvector U associated with a significant singular value of R corresponds to the wavefront, which must be re-emitted from the probe to focus selectively on the associated scatterer. The emitted wave is then optimally focused on each target through the medium.

Generally, in ultrasound imaging, an array of piezoelectric transducers is placed opposite the medium to be imaged (see part (a) of FIG. 3). These elements can emit/receive ultrasound waves independently over a wide spectral band. From a series of insonifications of the medium by all or part of this array, the echoes backscattered by the heterogeneities of the medium are recorded either by these same transducers or by other dedicated transducers. The signals thus recorded are then stored in a response matrix R (i.e. a reflection matrix, for example). The matrix can be acquired for different types of incident waves.

A simple acquisition sequence consists of emitting each incident wave with one element at a time (see part (a) of FIG. 3) and, for each emission, recording the field reflected by the medium on all the elements of the array (see part (b) of FIG. 3). This canonical base is used in the field of non-destructive testing, where it is known as “full matrix capture”. A matrix acquired in this way can be written mathematically as Rᵤᵤ(t)=[R(uₒᵤₜ,uᵢₙ,t)], where u is the position of the elements along the network, with the ‘in’ and ‘out’ indices indicating emission and reception respectively. The response matrix can also be acquired using beamforming (emission and/or reception with all elements in concert with appropriate delays between each element) in order to synthesise, for example, focused beams as achieved in B-mode imaging or plane waves to increase the image rate. In the latter case, for each plane wave the direction of which is given by the unit vector θᵢₙ, the reflected field recorded by the transducers is stored in a response matrix denoted R_{uθ}(t)=[R(uₒᵤₜ, θᵢₙ, t)]. In the following, we will consider ultrasound data acquired in the plane wave base during emission and in the transducer base during reception. However, this disclosure is general and can be applied to any acquisition base.

From the response matrix, an ultrasound image can be formed by coherently summing the recorded echoes from each focal point rₒᵤₜ, which then acts as a virtual detector within the medium. In practice, appropriate delays are applied to the recorded signals before they are summed. The images obtained for each incident wave are then added together coherently to produce a final image with improved contrast. This last operation subsequently generates a synthetic focus (i.e. a virtual source) on each focal point rᵢₙ=rₒᵤₜ. The composite image is thus equivalent to a confocal image that would be obtained by focusing the waves on the same point in emission and reception mode.

Matrix imaging involves decoupling the rᵢₙ and rₒᵤₜ focal points. A focused reflection matrix, Rᵣᵣ(δt)=[R(rᵢₙ, rₒᵤₜ, δt)], can be synthesised by summing and applying delay times to the IQ signals of the recorded response matrix. For the reflection matrix recorded in the plane wave base, the double focusing operation is expressed mathematically as follows:

$$R ( {r}_{in} , {r}_{out} , δ t ) = {\sum }_{{\hat{θ}}_{in} , {u}_{o u t}} R ( {\hat{θ}}_{in} , {u}_{out} δ t + {τ}_{i n} ( {\hat{θ}}_{in} , {r}_{in} ) + {τ}_{out} ( {u}_{out} , {r}_{o u t} ) ) \qquad [ Math 1 ]$$

with τᵢₙ and τₒᵤₜ the space-time delay laws to be applied at the input and output of matrix Rᵣᵣ(δt). These delay laws depend on our a priori knowledge of the sound speed distribution c(r) in the medium. For a homogeneous speed model (c(r)=c₀), τᵢₙ and τₒᵤₜ are expressed as follows:

$${τ}_{i n} ( {\hat{θ}}_{in} , {r}_{in} ) = {\hat{θ}}_{in} \cdot {r}_{in} / {c}_{0} \qquad [ Math 2 ]$$

$$and$$

$${τ}_{out} ( {u}_{out} , {r}_{o u t} ) = ‖ {u}_{o u t} - {r}_{o u t} ‖ / {c}_{0}$$

where (xᵢₙ, zᵢₙ) and (xₒᵤₜ, zₒᵤₜ) are the coordinates of focal points rᵢₙ and rₒᵤₜ.

This projection of matrix R(t) in a focused base can also be carried out using matrix products in the frequency domain. To do that, a first step is to create a temporal Fourier transform of the measured signals. A monochromatic reflection matrix R(f) is obtained at each frequency f. For an initial acquisition of R in the plane wave base, the projection of R_{uθ}(f) in the focused base is carried out using the following matrix product:

$${\tilde{R}}_{r r} ( f ) = {G}_{ur}^{†} ( f ) \times {\tilde{R}}_{u θ} ( ω ) \times {P}_{θ r}^{*} ( f ) \qquad [ Math 3 ]$$

in which matrices P_{θr}(f)=[P(θ, r, f)] and Gᵤᵣ(f)=[G(u, r, f)] are the transition matrices from the focused base to, respectively, the plane wave base and the transducer base. P_{θr} and Gᵤᵣ depend on our a priori knowledge of the sound speed distribution c(r) in the medium. For a homogeneous speed model, the coefficients of P_{θr} are given by:

$$P ( \hat{θ} , r , f ) = exp ( ik r \cdot \hat{θ} ) \qquad [ Math 4 ]$$

with k=2π f/c₀, the wave number. The coefficients of Gᵤᵣ correspond to the 2D or 3D Green functions of the wave equation in a homogeneous medium:

$${G}^{2 D} ( u , r ) = {G}_{2 D} ( u , r , f ) = - \frac{i}{4} {ℋ}_{0} ( k \vert u - r \vert ) , \qquad [ Math 5 ]$$

with [symbol] ₀ the 1^{st} order Hankel function the asymptotic expression of which is the following:

$${ℋ}_{0} ( 2 π f \vert u - r \vert / {c}_{0} ) \approx {e}^{3 i π / 4} \sqrt{\frac{2}{π}} \frac{{e}^{- {jk}_{0} \vert u - r \vert}}{\sqrt{{k}_{0} \vert u - r \vert}}$$

$${G}_{3 D} ( u , r , f ) = \frac{exp ( - i {k}_{0} \vert u - r \vert )}{4 π \vert u - r \vert} \qquad [ Math 6 ]$$

An inverse Fourier transform of Rᵣᵣ(f) can then be carried out to obtain focused reflection matrix Rᵣᵣ(St) in the time domain.

This disclosure relates to the use of matrix Rᵣᵣ(St) to detect and locate targets (i.e. target objects) in heterogeneous media.

### Detection of Buried Targets in Scattering Media: The Time Reversal Operator Decomposition Method

Response matrix R can be used to detect targets (i.e. target objects). Physically, this method makes it possible to focus selectively by iterative time reversal on each scatterer of a multi-target medium. Mathematically, it consists of an eigenvalue decomposition of the time reversal operator RR^{†} in the frequency domain. In the simple scattering regime and for spot targets, each clean space of RR^{†} is associated with a scatterer. A singular value decomposition (SVD) of R is carried out to compute the eigenspaces of RR^{†}:

$$\tilde{R} = U \times \sum \times {V}^{†} \qquad [ Math 7 ]$$

where E is a (diagonal) matrix of which the diagonal coefficients al are the singular values,
V and U are unitary matrices of which the column vectors Vᵢ and Uᵢ are the singular input and output vectors of R.

In the simple scattering regime, each eigenspace is associated with a target. The corresponding singular value σᵢ is proportional to the reflectivity of the target. If the matrix R_{uθ} is considered, the phase conjugate of the eigenvector, U_{u,i}, corresponds to the wavefront to be emitted from the probe to focus on the associated scatterer (see parts (d) and (e) of FIG. 3). An image of the target, Iᵢ^{(D)} is obtained by numerically re-propagating the associated eigenvector:

$${I}_{i}^{( D )} = {G}_{ur}^{†} \times {U}_{u , i} \qquad [ Math 8 ]$$

If the time reversal operator decomposition method is applied to the focused matrix Rᵣᵣ, the associated eigenvector U_{r,i} provides the image of the scatterer. Compared to a conventional ultrasound image, the image obtained by this method suffers from poor axial resolution. Indeed, the decomposition analysis of the time reversal operator is monochromatic. Axial resolution is therefore given by the probe's depth of field and not by the temporal resolution of the ultrasound signals.

The strength of the time reversal operator decomposition method lies in the fact that the bijective association between eigenspaces and scatterers remains valid in the presence of aberrations. However, target detection is only possible if the target has a higher scattering power than the ambient ultrasound speckle. In addition, in the multiple scattering regime, the reflection matrix must first be filtered to eliminate a large proportion of the multiple scattering before the time reversal operator decomposition method can be applied. In all cases, the target can be detected if the penetration depth remains limited to the mean free path of scattering [symbol] ₛ (average distance between two successive scattering events).

Finally, the bijective relationship between a clean space and a scatterer is only verified if the size of the target is smaller than one resolution cell of the imaging system. Otherwise, the rank of the matrix R is of the order of the number Q of resolution cells contained in the object. The target image is obtained by combining the first Q eigenspaces of Rt.

### Locating and Characterising Objects: The Generalised Polarisation Tensor

For electromagnetic waves, a mathematical method has been developed based on the monochromatic response matrix and the generalised polarisation tensor (GPT) of the object being probed (in this case, a dielectric inhomogeneity). When an antenna array surrounds the object, the target's GPT can be obtained accurately from the matrix RR^{†} by solving a system of linear equations. The result is a fast algorithm that identifies a target from a dictionary of pre-calculated GPT data. The location and shape of the object are characterised using an element in the dictionary after some rotation, scaling and translation. This dictionary matching procedure operates directly in the GPT data.

This disclosure is based on the concept of detecting and characterising targets using a pre-calculated or pre-recorded data dictionary but directly on the broadband reflection matrix. This means there is no system of equations to solve, making the method much simpler and more robust than the GPT approach. In addition, it does not require a transducer array surrounding the medium being studied. Finally, unlike the time-reversal operator decomposition method or the GPT approach, multispectral analysis has the advantage of axial resolution dictated by the probe's bandwidth rather than its depth of field. In addition, this allows optimum use to be made of the target's temporal (or spectral) response for its detection in complex media where its presence is generally masked by strong multi-scattering noise.

The Process According to Examples in this Disclosure

FIG. 3 schematically shows a diagram summarising the various steps of the matrix projection method based on a calibration measurement of a reflection matrix R₀(r₀) according to examples in this disclosure.

### Obtain a Reference Matrix Dictionary

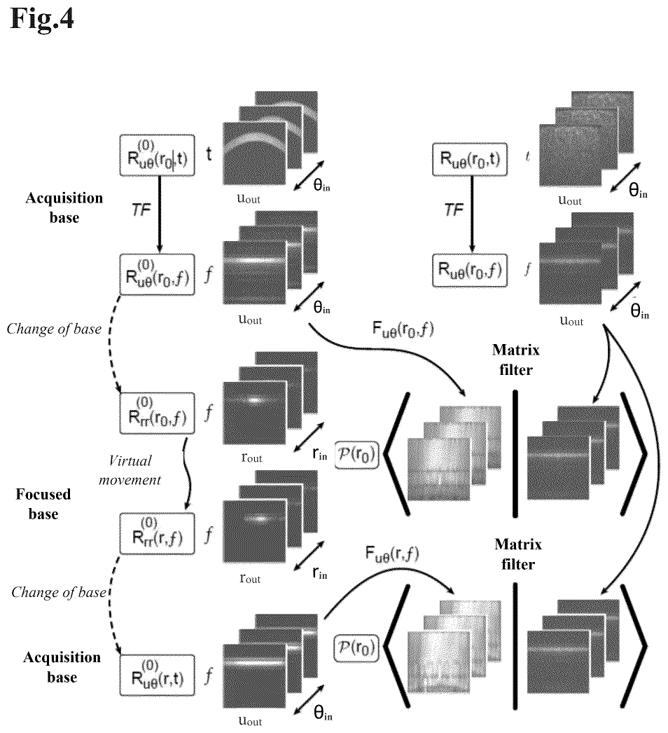

The first step consists in creating a reference matrix dictionary F(r, {qᵢ}) depending on position r of the target which it is sought to detect and on a set of parameters i which designate, for example, its size, shape, orientation, composition, local intensive parameters of the surrounding medium, such as temperature, pressure, composition, mechanical properties and/or the concentration of a compound in the surrounding medium, etc. In practice, this dictionary can be built using a calibration step consisting of experimentally measuring reflection matrix R₀(r, {qᵢ}) associated with the target for a set of positions r of the latter and a set of values of the parameters qᵢ. It can also be generated numerically using a wave propagation model from the probe to the object and a wave scattering model by the object. Finally, the two approaches can be combined by experimentally measuring reflection matrix R₀(r₀,q) for a target position r₀ and then deducing the value of F for each position r in the field of view (see FIG. 4). The same method or an artificial intelligence model can be used to determine the dependency of F(r, {qᵢ}) on other parameters qᵢ characterising the target.

Once this reference matrix dictionary has been compiled, the second step is to acquire reflection matrix R of the medium we are trying to characterise. The matrix filter then consists of carrying out a standard dot product between the measured matrix and the set of reference matrices F(r,q) in order to estimate the probability P(r,q) that the object is in position r and in state q. This dot product can be produced in the time domain,

$$P ( r , q ) = \frac{\vert \int dt Tr \{ R ( t ) {F}^{†} ( r , q , t ) \} \vert}{{[ \int dt {‖ R ( t ) ‖}_{2} \times \int dt {‖ F ( r , q , t ) ‖}_{2} ]}^{1 / 2}} \qquad [ Math 9 ]$$

or, in the frequency domain,

$$P ( r , q ) = \frac{\vert \int df Tr \{ R ( f ) {F}^{†} ( r , q , f ) \} \vert}{{[ \int dt {‖ R ( f ) ‖}_{2} \times \int dt {‖ F ( r , q , f ) ‖}_{2} ]}^{1 / 2}} \qquad [ Math 10 ]$$

with Tr{A} the trace of the matrix A, and ∥A∥₂=Tr{AA^{†}}.

If projector F (i.e. the reference matrix dictionary) is taken to be equal to matrix R₀, equations [Math 9] and [Math 10] correspond to a suitable and optimal filter for the object sought in matrix R. The result P(r, q) of the matrix projection method then corresponds to the coherent sum of the correlation coefficients between the elements of the R₀ and R matrices integrated over the bandwidth of the ultrasound signals. The RR₀^{†} operator differs from the time reversal operator RR^{†} is considered by the time reversal operator decomposition method. It reflects the ‘Scattering Invariant Mode’ (SIM) operator TT₀^{†} introduced by reference Pai et. al. in an emission configuration, see Pai, P., Bosch, J., Kohmayer, M. et al. Scattering invariant modes of light in complex media. Nat. Photonics 15, 431-434 (2021). The matrix T represents the response matrix between two transducer arrays placed on either side of the medium and the matrix τₒ is the reference matrix that would be obtained if the medium were homogeneous.

Depending on the configuration being studied and the calibration measurement quality, the F projector can take on more elaborate forms:
(I) by filtering the R₀ matrix in order to make the matrix filter more specific to the sought target relative to the surrounding medium;
(ii) by whitening its frequency spectrum;
(iii) by modifying the nature of the matrix filter. For example, the F projector can be built from an estimator of R₀₁ of the inverse matrix of R₀.

By taking F^{†}=R₀⁻¹, equations [Math 9] and [Math 10] correspond to an inverse filtering operation on matrix R. By way of illustration, several examples of F matrices will be considered in the following.

Above, the matrix projection method was defined mathematically (Eqs. [Math 9] and [Math 10]). In what follows, its advantages will be illustrated in the context of two examples for the detection and location of targets and for quantitative ultrasound imaging of biological tissues.

### Detection and Location of Targets Buried in a Scattering Medium

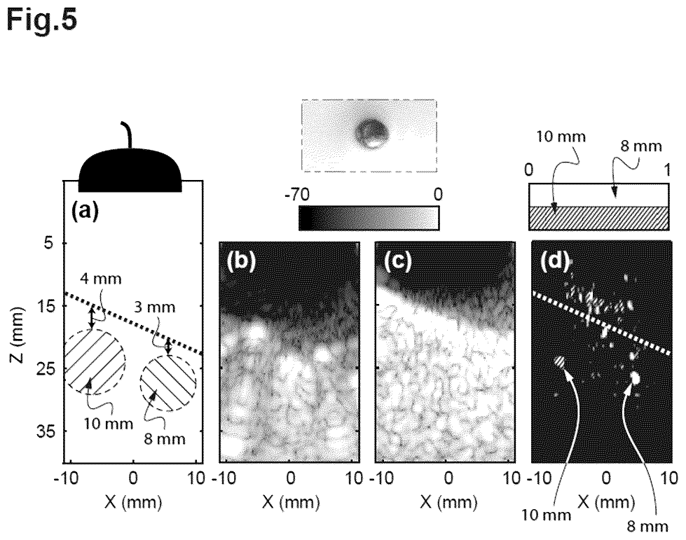

FIG. 5 schematically shows a proof of concept according to a first experiment comprising matrix imaging of steel spheres in highly scattering granular media according to examples in this disclosure. Part (a) of FIG. 5 schematically shows an experimental set-up: A matrix probe measures reflection matrix R associated with the medium of interest, two steel spheres buried in a particularly scattering granular medium. Parts (b) and (c) of FIG. 5 schematically show an ultrasound image of the two spheres, respectively, in the absence and presence of the granular medium. Part (d) of FIG. 5 schematically shows a position likelihood map for each intruder resulting from the adapted matrix filter.

For the first experiment, the experimental set-up consists of a 2D matrix probe placed in front of a highly scattering granular suspension made up of a random stack of 315 μm diameter glass beads immersed in water. A frequency filter was applied to reduce the bandwidth over a domain where the wave's transport properties can be considered relatively constant. Using emission measurements of the coherent pulse, we estimate the longitudinal phase speed c_{φ}˜1.6 mm/ps and the free scatter path [symbol] ₃ is of the order of 1 mm in the studied bandwidth. [symbol] ₛ corresponds to the average distance between two successive scattering events experienced by the wave. The objects to be imaged are two steel spheres, 8 mm and 10 mm in diameter, with their centres buried at z˜7 [symbol] ₛ and 9 [symbol] ₛ below the surface of the medium (see part (a) of FIG. 5). These imaging conditions are therefore particularly disadvantageous because the simply scattered wave is exponentially attenuated by the scattering of the granular medium by a factor of exp(−z/ [symbol] ₛ). Part (c) of FIG. 5 shows the confocal image of the whole medium.

The presence of the two targets was not revealed due to the predominance of a multiple scattering background. Very advantageously, the matrix projection method described above can be used to build a likelihood map for each target (see part (a) of FIG. 5) and their exact positions can be found unambiguously.

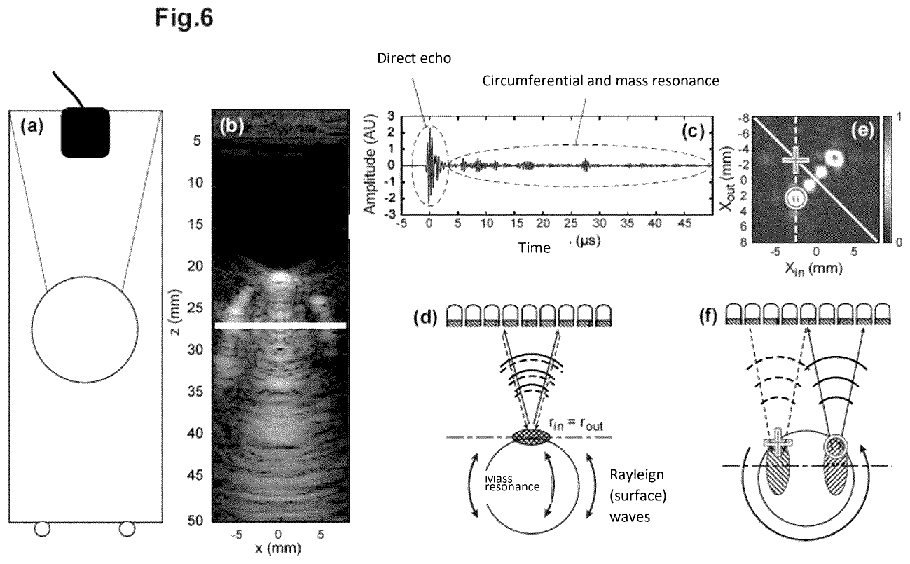

FIG. 6 schematically shows a device and method for obtaining a reference matrix R₀ measured experimentally in the context of the first experiment according to examples in this disclosure. The device and method can be used to detect and locate targets buried in a scattering medium in accordance with this disclosure. This experiment uses a reference matrix R₀ measured for a position of the steel sphere (φ=8 mm). Part (a) of FIG. 6 schematically shows an experimental set-up used to record R₀. Part (b) of FIG. 6 schematically shows an image taken in B-mode (B-“brightness” scan of the ultrasound image). Part (c) of FIG. 6 schematically shows the time dependence of the confocal signal at the sphere cap. Part (d) of FIG. 6 schematically shows the resonance of the volume and surface waves explaining the long time tail of the signal visible in parts (b) and (c). Part (e) of FIG. 6 schematically shows the matrix R₀ in the focused base at a depth z=27 mm of the ultrasound image [horizontal line on part (b)]. Part (f) of FIG. 6 schematically shows the surface waves that explain the off-diagonal signal in part (e).

This advantageous result described in the context of FIG. 5 was obtained by measuring, in a first calibration step, the reflection matrix associated with each of the spheres for a given position (see part (a) of FIG. 6). These matrices can therefore form a reference matrix dictionary. Using a process described below, we can virtually move the object in the field of view and determine the spatial evolution of R₀(r,ϕ). The complex space-time signature of the spheres is illustrated in FIG. 6, which shows the case of the 8 mm steel sphere in water.

Its confocal signal has a long reverberation time (see part (c) of FIG. 6) due to the Mie resonances (volume waves) (see part (d) of FIG. 6) and Rayleigh (surface) resonances (see part (f) of FIG. 6) that such a sphere can generate.

The reflection matrix also has a complex spatial signature highlighted by high off-diagonal energy in the focused base (see part (e) of FIG. 6). In particular, to highlight the specificity of the target's space-time signature compared to the signature of the surrounding objects, a temporal filter can be applied to the reflection matrix to keep only the echoes recorded at long times, corresponding to the surface and volume waves propagating on and in the target.

This complexity of the space-time signature of each sphere can be quantified by the number of spatial and temporal degrees of freedom, Nₛ and Nₜ, contained in the signature. Nₛ is equal to the effective rank of matrix R₀, i.e. approximately the number of transverse resolution cells occupied by the target. Nₜ is equal to the product of the target's reverberation time and the frequency bandwidth. In this case, the large size of the targets (Nₛ˜20) combined with their long temporal tail (Nₜ˜20)) results in a high number of space-time degrees of freedom. By compressing the signal associated with each target into a single point, the matrix projection developed here enhances the single-over-multiple scattering ratio by a factor of M=Nₛ×Nₜ˜400. This considerable gain compensates for the exponential attenuation of simply scattered waves in the granular medium [ ][exp(−z/ [symbol] ₛ)˜2−5×10⁻²]]. A detection of each target and their location are obtained as if the granular medium had been made suddenly homogeneous (see part (d) of FIG. 5).

### Detection and Location of a Lesion Marker Embedded in Ultrasound Speckle

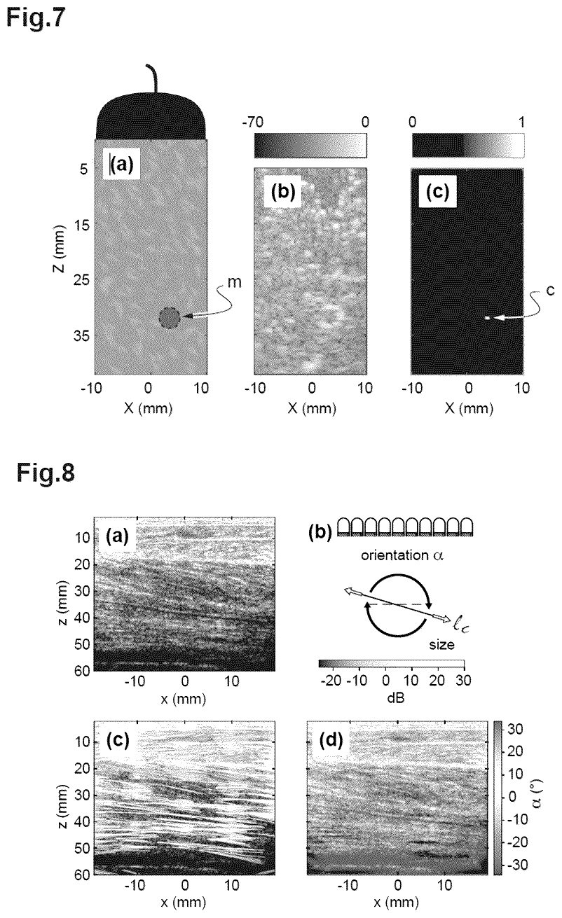

FIG. 7 schematically illustrates a method for detecting and locating a lesion marker in ultrasound speckle according to a second experiment carried out in accordance with examples in this disclosure. Part (a) of FIG. 7 schematically shows a 3D spherical lesion marker m in an experimental set-up. Part (b) of FIG. 7 schematically shows the ultrasound image obtained in B mode. Part (c) of FIG. 7 schematically shows a position likelihood map c for the marker resulting from the adapted matrix filter.

A second experiment involved detecting and locating a lesion marker used to mark biopsy sites and suspicious lesions in breast tissue. For this experiment, the marker is positioned in foam submerged in water. This foam generates typical ultrasound speckle, for example for breast tissue. The presence of the marker is difficult to detect on the conventional ultrasound image acquired using the 2D probe (see FIG. 7). In contrast, this disclosure implemented using a matrix projector uses the marker's matrix signature to detect it with excellent contrast and locate it with an accuracy well below the diffraction limit.

### Imaging Fibrous Tissue Anisotropy

FIG. 8 schematically shows an example of the matrix imaging of a calf, in which the reference matrix dictionary is composed entirely synthetically in accordance with this disclosure.

Part (a) of FIG. 8 schematically shows an ultrasound image of the calf in dB (c₀=1580 m/s). Part (b) of FIG. 8 schematically shows a numerically simulated experimental configuration (homogeneous sound speed model c₀) to create a matrix dictionary R₀(r,{α, [symbol] _{c}}) associated with a mirror of orientation a and size [symbol] _{c}. Part (c) of FIG. 8 schematically shows the orientation and local size of the fibres, α_{c}^{(apt)}(r) and [symbol] (r) estimated by the adapted matrix filter for transverse positions x={−10;0;10} mm. Part (d) of FIG. 8 schematically shows a mapping of fibre orientation superimposed on the ultrasound image of the calf.

In addition to detecting and locating targets, the adapted matrix filter can be used for quantitative imaging of tissues using ultrasound echography. For example, the anisotropy of the fibrous tissues that typically make up muscles can be mapped. This measurement is particularly relevant for diagnosing neuromuscular diseases or diseases affecting the myocardium. In elastography, fibrous media also generate shear wave speed anisotropy. Detailed knowledge of this anisotropy is therefore required to map tissue stiffness correctly based on shear wave propagation films.

This third experiment was carried out on a calf, the fibres of which are partly visible in the ultrasound image shown in part (a) of FIG. 8. Reflection matrix R is recorded using a linear transducer array (N=256 elements spaced 0.2 mm apart, centre frequency 7.5 MHz and bandwidth [2.5;9.5] MHz). The illumination base consists of 101 plane waves of which the angle of incidence varies from −25° to 25°. For each insonification, the reflected field is recorded by all the transducers.

To quantitatively image the fibrous tissue, the adapted matrix filter was applied to matrix R, considering here, as the reference object, a mirror of constant reflectivity of which the parameters qᵢ are here the orientation a relative to the probe and the characteristic size [symbol] _{c} (see part (b) of FIG. 8). A reference matrix dictionary F(r,{a, [symbol] _{c}}) associated with this object is built using a numerical calculation described below.

The adapted matrix filter can be used to calculate a likelihood index P(r,{a, [symbol] _{c}}) relative to parameters a and [symbol] _{c} at each point r in the image (see Eq. 10). The maximum value of this quantity gives the orientation α_{c}^{(opt)}(r) and the characteristic size [symbol] (r) of the scatterers at each point in the image:

$$\{ {α}_{c}^{( opt )} ( r ) , {ℓ}_{c}^{( opt )} ( r ) \} = {arg max}_{α , {ℓ}_{c}} [ P ( r , \{ α , {ℓ}_{c} \} ) ] \qquad [ Math 11 ]$$

The size and orientation of the fibres determined in this way are shown for different transverse positions in part (c) of FIG. 8. A detection threshold was applied a priori by only representing the result of the matrix filter with an associated likelihood index greater than 10⁻³. The fibre orientation map is also superimposed on the ultrasound image in part (d) of FIG. 8. Very good agreement is found between the visual appearance of the fibres on the ultrasound image and their orientation as determined by the adapted matrix filter.

Note that this approach can be considered as a generalisation of the specular beamforming developed e.g. by Rodriguez-Morales et al, see A. Rodriguez-Molares, A. Fatemi, L. Løvstakken and H. Torp, “Specular Beamforming,” in IEEE Transactions on Ultrasonics, Ferroelectrics, and Frequency Control, vol. 64, no. 9, pp. 1285-1297, Sept. 2017, doi: 10.1109/TUFFC.2017.2709038.

The latter approach is in fact a suitable filter for a mirror of infinite size. Specular beamforming is therefore suitable for detecting and locating large objects in ultrasound imaging. On the other hand, optimising the characteristic size of the scatterer means that the filter is better suited to fibrous media. It is also an important parameter for locally adapting beamforming to the different types of encountered fibres and obtaining a better quality ultrasound image. The formed image is, for example, the maximum of the observable P measured for each pixel. Apart from that, other objects can be simulated synthetically, for example a needle inserted into the tissue.

There is a link between the adapted matrix filter and the analysis of the reflection matrix in the plane wave base developed in the context of non-destructive testing for the purpose of characterising anisotropic media. However, this approach does not make it possible for the anisotropy of the fibres to be resolved laterally; only the axial evolution of the anisotropy can be determined. The spatial correlation technique of the backscattered field can only determine the orientation of the fibres in a plane parallel to the probe.

Matrix projection of ultrasound data is a very general method that can be applied to the imaging of a large number of medium parameters. The parameters considered here are, for example, the size and orientation of the scatterers, but their shape or composition can also be parameters that can be mapped using this method. A necessary condition, however, is the creation of a sufficiently complete and accurate dictionary of the structures being studied. To that end, the following section describes the different ways in which such a dictionary can be compiled.

### Creation of a Reference Matrix Dictionary

There are various ways of numerically generating a reference matrix dictionary F(r,q). A first approach involves measuring, in a first calibration step, reflection matrix R₀(r₀,q) associated with the target alone, for one of its positions, and then generating a reference matrix dictionary F(r,q) for each point r in the field of view. An alternative strategy is to calculate the set of reference matrices F(r,q) analytically.

### Numerical Emulation of a Dictionary Based on an Experimental Measurement

Part (a) of FIG. 6 shows the experimental configuration for the R₀ calibration measurement carried out as part of the experiment to detect targets buried in a scattering medium. The matrix array of 1024 transducers located at z=0 is used to independently probe each target placed at a position rₒ and immersed in water. The associated reference matrix, R_{uθ}(r₀, t) is recorded in the time domain between the input plane wave base (θᵢₙ) and the output transducer base (uₒᵤₜ). It thus contains all the signals measured by each of the transducers identified by their coordinates uₒᵤₜ for a set of plane wave illuminations propagating in direction θᵢₙ. Note that this choice of illumination and reception bases can be arbitrary and that the reference matrix could very well be measured using another sequence of illuminations, in the focused base for example. In addition, intermediate steps can be considered, such as temporal filtering of the measured reference matrix, spectrum whitening, etc.

Once this reference matrix has been measured, a Fourier transform is applied to it in the time domain. Thus, reference matrix, R_{uθ}⁽⁰⁾(r₀,f) in the frequency domain is obtained. A translation of the target can then be simulated numerically in a new position r in the medium. This operation can be carried out in different bases, for example the focused base. The first step is to project R_{uθ}⁽⁰⁾(r₀,f) in this base (Eq. [Math 3]) and then generate a new matrix R_{uθ}⁽⁰⁾(r,f) for a new target position r using the coefficients of Rᵣᵣ⁽⁰⁾(r,f) such that:

$${\tilde{R}}_{0} ( {r}_{in} , {r}_{out} , r ) = {\tilde{R}}_{0} ( {r}_{in} - Δ r , {r}_{out} - Δ r , {r}_{0} ) with Δ r = r - {r}_{0} . \qquad [ Math 12 ]$$

Depending on the matrix acquisition base, it may also be necessary to work in the transducer base to limit calculation time and/or memory use. A matrix Rᵤᵤ⁽⁰⁾(r,f) can be generated for each virtual target position r from its measurement by rₒ by decomposing the displacement Δr along its transverse projections Δp and axial projections Δz. Transverse displacement is achieved by translating the transducer base:

$${\tilde{R}}_{0} ( {u}_{in} , {u}_{out} , \{ ρ , {z}_{0} \} ) = {\tilde{R}}_{0} ( {u}_{in} - Δρ , {u}_{out} - Δρ , \{ {ρ}_{0} , {z}_{0} \} ) \qquad [ Math 13 ]$$

with p and p₀ are the transverse coordinates of points r and r₀.

Axial displacement, on the other hand, requires data to be propagated from the zₒ plane to the z plane at the input and output of the matrix Rᵤᵤ⁽⁰⁾:

$${\tilde{R}}_{uu}^{( 0 )} ( r ) = {G}_{u ρ} ( z ) \times {G}_{u ρ}^{†} ( {z}_{0} ) \times {\tilde{R}}_{uu}^{( 0 )} ( \{ ρ , {z}_{0} \} ) \times {G}_{u ρ}^{*} ( {z}_{0} ) {G}_{u ρ}^{T} ( z ) \qquad [ Math 14 ]$$

where the symbol T denotes the matrix transposition operation.

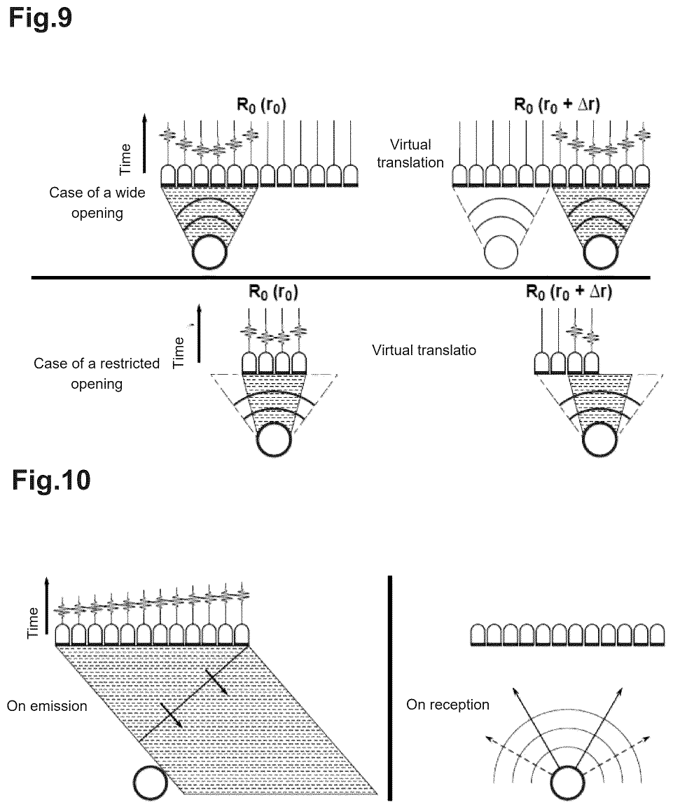

FIG. 9 schematically shows the virtual translation of the target object position according to examples in this disclosure. The upper part (“Case of a large opening”) of FIG. 9 schematically shows that by carrying out this translation in the transducer base or in the focused base, it is possible to virtually move the object as long as the field support associated with the target is contained within the opening of the transducer array. The lower part (“Case of restricted opening”) of FIG. 9 schematically shows that for a lateral position of the target approaching the ends of the transducer array, a lateral translation of the ultrasound signals is no longer adequate.

Reflection matrix R₀(r) can also be generated from matrix R₀(r₀) from the Fourier base. The first step is therefore to project experimentally acquired matrix R₀(r₀) into the plane-wave base at both input and output. For an R₀(r₀) matrix recorded using a plane-wave illumination sequence, only an output projection into the Fourier base is required:

$${\tilde{R}}_{θθ}^{( 0 )} ( {r}_{0} ) = {P}_{θ u} \times {\tilde{R}}_{u θ}^{( 0 )} ( {r}_{0} ) \qquad [ Math 15 ]$$

In the case of a matrix acquired in the canonical base (transducers), the projection must be carried out on the input and output of the matrix: Note that the canonical base is only an illustrative example.

$${\tilde{R}}_{θθ}^{( 0 )} ( {r}_{0} ) = {P}_{θ u} \times {\tilde{R}}_{u θ}^{( 0 )} ( {r}_{0} ) \qquad [ Math 16 ]$$

Once the matrix R₀(r₀) has been projected into the plane wave base, its virtual displacement to position r can be carried out as follows:

$$\tilde{R} ( {\hat{θ}}_{out} , {\hat{θ}}_{in} , r ) = P ( {\hat{θ}}_{out} , r ) P * ( {\hat{θ}}_{out} , r ) \tilde{R} ( {\hat{θ}}_{out} , {\hat{θ}}_{in} , r ) P * ( {\hat{θ}}_{in} , {r}_{0} ) P ( {\hat{θ}}_{in} , r ) = P ( {\hat{θ}}_{out} , Δ r ) \tilde{R} ( {\hat{θ}}_{out} , {\hat{θ}}_{in} , r ) P ( {\hat{θ}}_{in} , Δ r ) , \qquad [ Math 17 ]$$

This equation involves applying input and output phase ramps to the data projected into the plane wave base. Depending on the position r of the object, not all the plane waves emitted from the probe will reach it, just as the backscattered echoes arriving at the transducers can only come from a limited opening (see FIG. 10).

FIG. 10 schematically shows the emission and reception of a plane wave associated with an angle of incidence from which the data must be filtered in matrix R₀ according to examples in this disclosure. The left part of FIG. 10 schematically shows a plane wave associated with an angle of incidence from which the data must be filtered in matrix R₀. The right part of FIG. 10 schematically shows that on reception, the plane waves associated with the arrows pointing towards the transducer array and the other arrows pointing outside the transducer array must be respectively kept and filtered in the measured R₀ matrix.

Any signal corresponding to extreme angles, which can be determined depending on the geometry of the probe and the position of the object, is therefore of little interest and can be assimilated to noise, which therefore needs to be filtered in an implementation option.

Whichever base is focused, the transducer base or the Fourier base, equations 12, 13 and 14 are based on an assumption of invariance by translation of the focusing process (see FIG. 9). This last hypothesis is only verified if the entire field associated with the target is measured by the transducer array:

$$\sqrt{{x}^{2} + {y}^{2}} < \frac{A}{2} - z tan β \qquad [ Math 18 ]$$

with β the opening angle of the probe imposed by its directivity and A is its width.

The choice of the base in which the virtual translation is to be carried out is dictated by considerations of time and calculation costs. In near-real-time mode, for example, it may be possible to choose to carry out all the operations from the acquisition base of the Rₒ and R reflection matrices in order to limit the number of matrix operations to be carried out.

### Reference Matrix Optimisation

### Specificity of the Reference Matrix Relative to the Surrounding Medium

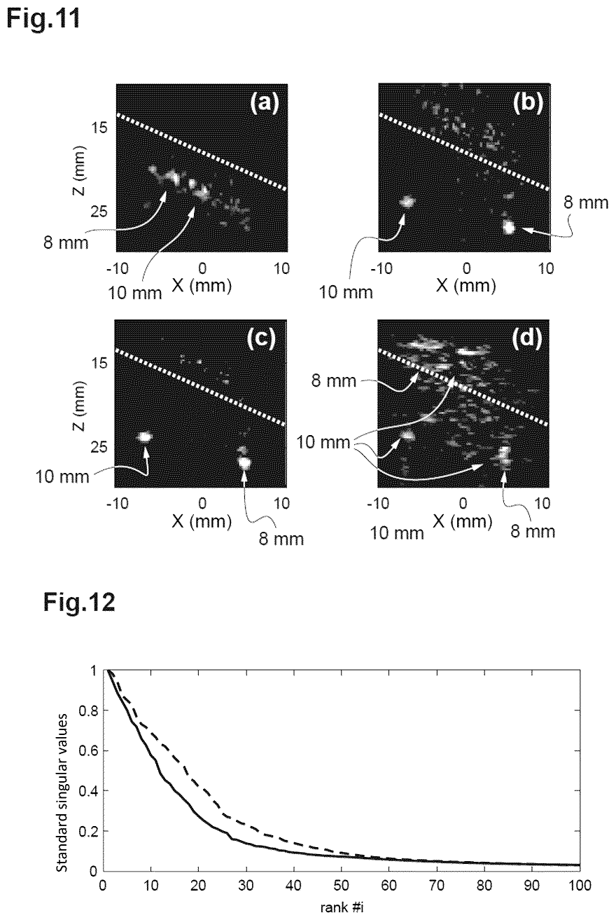

FIG. 11 schematically shows position likelihood maps for each target resulting from the matrix projection defined by equation 10 according to examples in this disclosure. Part (a) of FIG. 11 schematically shows an adapted filter with F(r, Φ)=R₀(r, Φ). Part (b) of FIG. 11 schematically shows an adapted filter after removal of the specular echo of the targets with F(r, Φ)=R_{S}(r, Φ) [see Eq. 19]. Part (c) of FIG. 11 schematically shows an adapted filter with F(r, Φ)=Rₛ(r, Φ) [see Eq. 20]. Part (d) of FIG. 11 schematically shows an inverse filter with P(r, Φ)=R_{S}⁻¹(r, Φ) [see Eq. 22]. Note that the frequency filter applied to the ultrasound data here is broadband (Hann window, [0;6] MHz).

In the case of the experiment to detect targets in a granular medium, one step consists in first filtering the specular echo of the targets in R₀(r, Φ). Without this prior filtering, the image obtained is dominated by the interface echo of the granular medium (see part (a) of FIG. 11). The specular echo is not specific to the target and will also enhance the interface echo of the granular medium which dominates the signals recorded in the matrix R.

There is therefore an advantage in building reference matrices F(r, Φ) from the echoes most specific to each target. In this illustrative example, these are the multiple reflection echoes within them and the surface wave echoes previously highlighted in FIG. 6. In practice, this operation can be carried out by temporal windowing of the measured ultrasound signals (see part (b) of FIG. 6):

$${R}_{S} ( {r}_{0} , Φ , t ) = {R}_{0} ( {r}_{0} , Φ , t ) H ( t - {t}_{0} - δ t ) \qquad [ Math 19 ]$$

where H is the Heaviside function, to is the arrival time of the direct echo from the target and δt=Δf⁻¹ is the temporal resolution of the measured ultrasound signals.

The specific reference matrix Rₛ is then virtually translated and used to build a suitable image P(r, Φ) of the sought targets [see Eq. 9 with F=R_{S}^{†}]. In this example, the result is shown in part (c) of FIG. 11. Advantageously, the interface echo from the granular medium is much less intense, the two targets are detected with good contrast and the position of their centre is found with excellent accuracy.

### Frequency Spectrum Whitening

A second step consists in whitening the frequency spectrum of the matrix R_{S}, such that:

$${\overline{R}}_{s} ( r , Φ , f ) = {\tilde{R}}_{s} ( r , Φ , f ) / {‖ {\tilde{R}}_{s} ( r , Φ , f ) ‖}_{2}^{1 / 2} \qquad [ Math 20 ]$$

By considering this family of normalised matrices R_{S} as the reference dictionary F in equation 10, a new map P(r, Φ) is obtained in part (c) of FIG. 11. The frequency whitening operation makes the most of the temporal degrees of freedom highlighted by part (c) of FIG. 6 and improves the contrast between the targets and the noise associated with the surrounding medium.

### Reverse Filter

The previous examples were limited to a filter adapted from the R matrix, whereas an inverse filter should in theory be able to maximise the contrast of the two targets. The matrix inversion process is poorly conditioned and extremely sensitive to noise. A regularisation method can be used to optimise the inversion process. To do that, the singular value decomposition of R_{S} must first be calculated:

$${\overline{R}}_{s} ( f ) = {\sum }_{i = 1}^{N} {σ}_{i} ( f ) {U}_{i} ( f ) {V}_{i}^{†} ( f ) \qquad [ Math 21 ]$$

The spectrum of singular values is shown in FIG. 12 for both targets. A large number of singular values exceed the noise level.

FIG. 12 schematically shows a spectrum of singular values σᵢ of matrices R_{S} associated with spheres according to examples in this disclosure, for example spheres of diameter Φ=8 mm and Φ=10 mm at frequency f=2.5 MHz after time windowing to only keep the multiple reflection and surface wave echoes specific to the target.

The set of eigenspaces associated with these singular values forms the signal subspace associated with the target. Rank Q of this subspace increases as the number of resolution cells in the target increases. An estimator R_{S}⁻¹(f) of the inverse matrix of k(f) can be calculated by inverting the first Q singular values σᵢ of the signal subspace of R₃ and cancelling that of the noise subspace:

$${\hat{R}}_{s}^{- 1} ( f ) = {\sum }_{i = 1}^{Q} {σ}_{i}^{- 1} ( f ) {U}_{i} ( f ) {V}_{i}^{†} ( f ) \qquad [ Math 22 ]$$

The resulting dictionary, F(r,f)=R_{S}⁻¹(r,f) can then be used to build an image of the probability of the target's presence [see Eq. 10]. The result is shown in part (d) of FIG. 11 for Q=20. Admittedly, the resolution of the echoes associated with each target is finer than for the adapted filter in part (c) of FIG. 11.

However, this improvement in resolution comes at the cost of significant false alarms upstream of the targets and cross-talk (i.e. the interference of a first signal with a second) between them. These artefacts are linked to the sensitivity of the inversion process to experimental noise.

In one variant, a Tikhonov-type regularisation can be applied to improve the robustness of the method. Matrix F(r₀,f) is expressed in this case as:

$$\tilde{F} ( f ) = {\sum }_{i = 1}^{Q} \frac{{σ}_{i} ( f )}{{σ}_{i}^{2} ( f ) + {b}^{2}} {V}_{i} ( f ) {U}_{i}^{†} ( f ) \qquad [ Math 23 ]$$

with b is the estimated noise level on the spectrum of singular values.

### Numerical Generation of a Dictionary

The reference matrix dictionary can also be built entirely synthetically, i.e. obtained by calculation and simulation, without experimental measurement. To illustrate, the example of muscle anisotropy imaging can again be used as an example. The results shown in FIG. 8 were obtained by numerically calculating the reference matrix F(r₀) using the following matrix product:

$${\tilde{R}}_{uu}^{( 0 )} ( {r}_{0} ) = {P}_{θ u}^{†} \times {P}_{θ r} \times Γ ( r , α , {ℓ}_{c} ) \times {P}_{θ r}^{T} \times {P}_{θ u}^{*} \qquad [ Math 24 ]$$

where F(r, α, [symbol] _{c}) is the reflectivity matrix of a plane mirror of tilt a and size [symbol] _{c} [see part (b) of FIG. 8] centred at r in real space.

Γ(r, α, [symbol] _{c}) is a diagonal matrix of which the coefficients γ(r) correspond to the reflectivity of the mirror of which the response is being simulated.

In practice, it is advantageous for the real space to be sampled using a grid spacing much smaller than the diffraction limit (˜λ/5). The grid defined in real space is not the same as that of the focused base, of which the spatial sampling is of the order of λ/2.

The results shown in FIG. 8 were obtained using equation 10, using as reference matrices F(r, α, [symbol] _{c}) the simulated matrices R₀(r, α, [symbol] _{c}). Note that, in the case of a plane mirror, the number of non-null singular values of R₀(r, α, [symbol] _{c}) is equal to the number of resolution cells contained in the object and that these eigenvalues are all degenerate. The inverse filter [F(r, α, [symbol] _{c})=R₀⁻¹(r, α, [symbol] _{c})] therefore would give exactly the same result.

All of these and other embodiments as described above are by way of non-limiting example only, and may be combined and/or modified within the scope of this disclosure.

1. A method of characterizing a target object in a medium, the method comprising:
obtaining a reference matrix associated with the target object;
obtaining a reflection matrix of the medium; and
projecting the reflection matrix onto the reference matrix to estimate a probability that the object is in a state.

2. A method according to claim 1, wherein the target object comprises at least one of:
a predefined element of the medium comprising a tissue of the medium, and an external object inserted into the medium.

3. A method according to claim 1, wherein the state of the target object comprises at least one of:
a position of the external object in the medium,
an orientation of the object in the medium,
a shape and/or structure of the object,
a size of the object,
a composition of the object, and
one or more local parameters of the surrounding medium, the one or more local parameters comprising at least one of temperature, pressure, composition, mechanical properties and the concentration of a compound in the surrounding medium.

4. A method according to claim 1,
wherein obtaining the reflection matrix comprises obtaining reflection matrices measured at different times, and
wherein each of the reflection matrices is projected onto the reference matrix to measure the dynamics of the state of the object.

5. The method according tom claim 1, wherein:
obtaining a reference matrix comprises obtaining a reference matrix dictionary associated with the target object, and
projecting the reflection matrix comprises projecting the reflection matrix onto the set of reference matrices to estimate a probability that the object is in a state.

6. A method according to claim 1, wherein projecting the reflection matrix comprises applying a matrix filter to reflection matrix.

7. A method according to claim 6, wherein the matrix filter comprises one of:
a standard dot product between reflection matrix and the set of reference matrices, and
an artificial intelligence model.

8. The method according to claim 1, wherein the reference matrices of the dictionary depend on a state of the target object, the state comprising at least one parameter from among: position, orientation, shape and/or structure, size, composition.

9. The method according to claim 1, wherein obtaining the reference matrix comprises at least one of:
measuring a first reference matrix corresponding to a reflection matrix for a first value of the state of the target object, and
measuring a first set of reference matrices associated with several states of the target object.

10. A method according to claim 1, wherein obtaining a reference matrix comprises:
numerically generating a second reference matrix for a second state of the target object based on a first measured reference matrix for a state of the target object.

11. A method according to claim 10, wherein the second reference matrix is numerically simulated based on the first reference matrix by translation of the target object to a new state being the position of the object in a focused base, the transducer base, or the plane wave base.

12. A method according to claim 9, wherein the reference matrix dictionary comprises the first and second reference matrices.

13. A method according to claim 10, wherein the reference matrix dictionary is optimized using at least one of:
adapting the matrix filter depending on the first reference matrix to make the matrix filter more specific to the sought target object relative to the surrounding medium;
optimizing the dictionary's frequency spectrum to improve the contrast between the target object and a noise associated with the medium; and
building the dictionary based on an estimator of the inverse matrix of the first reference matrix.

14. A method according tom claim 1, wherein at least one of the first reference matrix and the reflection matrix is obtained using an ultrasound imaging method.

15. A method according to claim 1, wherein obtaining at least one of the first reference matrix and the reflection matrix comprises:
generating a series of incident waves in a medium, via an array of transducers, said series of incident waves being an emission base; and
generating an experimental reflection matrix defined between the input emission base and an output reception base;
determining a focused reflection matrix in a focused base, the focused reflection matrix comprises responses of the medium between an input virtual transducer of spatial position and an output virtual transducer of spatial position, the responses of the virtual output transducer being taken at a time instant offset by an additional delay relative to a time instant of the responses of the virtual input transducer, wherein the waves comprise ultrasound waves.

16. A method according to claim 1, wherein obtaining a reference matrix comprises building a reference matrix dictionary entirely synthetically by numerically simulating:
a first reference matrix of a virtual reflector for a predefined state, and
a second reference matrix for a second state of the target object, depending on the first reference matrix R₀(q).

17. A method of one of recognising and finding a target object in a medium, the method comprising a method according to claim 1.

18. A method of generating a map of a medium comprising a target object, the method comprising:
the method according to claim 1, and
generating the map depending on the estimated state of the target object.

19. A method according to claim 18,
wherein the map of the medium consists of an image of the medium and an identification of the superimposed target object.
